(19) United States
(12) Patent Application Publication
(10) Pub. No.: US 2023/0190574 A1
(43) Pub. Date: Jun. 22, 2023

(54) Systems And Methods For Therapeutic Sound Treatment

(71) Applicant: Jonathan William Christman, Edgewater, FL (US)

(72) Inventor: Jonathan William Christman, Edgewater, FL (US)

(21) Appl. No.: 18/064,568

(22) Filed: Dec. 12, 2022

Related U.S. Application Data
(60) Provisional application No. 63/290,346, filed on Dec. 16, 2021.

(51) Int. Cl. A61H 23/02; G06F 3/16; G06F 3/0482

(52) U.S. Cl. CPC A61H 23/0236; G06F 3/16; G06F 3/0482; A61M 2021/0027

(57) ABSTRACT

Described herein are methods and systems for providing therapeutic sound treatment to the human body. The present methods and systems may comprise a seating apparatus, a computing device, and a plurality of output devices. The computing device may provide a user interface allowing selection of one or more symptoms. The one or more symptoms may be associated with a sound frequency (or frequencies) and a duration(s) and/or pattern for output of the sound frequency (or frequencies). The computing device may cause the plurality of output devices to output the sound frequency (or frequencies) associated with the selected one or more symptoms, which may provide health benefits for the user, such as alleviating the selected one or more symptoms.

### CROSS-REFERENCE TO RELATED PATENT APPLICATION

This application claims priority to U.S. Provisional Patent Application No. 63/290,346, filed on Dec. 16, 2021, the entirety of which is incorporated by reference herein.

### SUMMARY

It is to be understood that both the following general description and the following detailed description are exemplary and explanatory only and are not restrictive. Described herein are methods and systems for providing therapeutic sound treatment to the human body. The present methods and systems may comprise a seating apparatus, a computing device, and a plurality of output devices that may be controlled via the computing device and a control device. The computing device may provide a user interface allowing selection, by a user, of one or more symptoms. The one or more symptoms may be associated with a sound frequency (or frequencies) and a duration(s) and/or pattern for output of the sound frequency (or frequencies). The computing device, via the control device, may cause the plurality of output devices to output the sound frequency (or frequencies) associated with the selected one or more symptoms, which may provide health benefits for the user, such as alleviating the selected one or more symptoms. Other examples are possible as well. Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of the present description serve to explain the principles of the methods and systems described herein:

FIG. 1 shows an example system;

FIG. 2 shows an example side-view of the example system;

FIG. 3 shows an example side-view of the example system

FIG. 4A shows a top view of an example control device;

FIG. 4B shows a rear view of the example control device;

FIG. 5 shows a block diagram of the example system;

FIG. 6 shows an example user interface;

FIG. 7 shows an example flow diagram;

FIG. 8 shows an example system;

FIG. 9 shows a flowchart for an example method; and

FIG. 10 shows a flowchart for an example method.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memristors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

Described herein are methods and systems for providing therapeutic sound treatment to the human body to, for example, alleviate symptoms of physical and/or mental illness(es). There have been several attempts to provide therapeutic sound treatment to the human body to alleviate both physical and mental illness symptoms, but these existing systems and methods require the body to come into contact with an apparatus producing vibrations. Indeed, it is the vibrations that provide the therapeutic effect to the users of these existing devices that employ existing systems and methods. Accordingly, the existing systems and methods are limited to specific apparatuses that enable the transmission of vibrations to users lying on support structures so to come into contact with the produced vibrations.

The present methods and systems represent improvements to the existing systems and methods described above. For example, the present methods and systems may provide therapeutic effects/benefits to users without requiring specific apparatuses that produce vibration/movement. The present methods and systems may comprise a seating apparatus, a computing device, and a plurality of output devices that may be controlled via the computing device and a control device. The computing device may provide a user interface allowing selection, by a user, of one or more symptoms. The one or more symptoms may be associated with a sound frequency (or frequencies) and a duration(s) and/or pattern for output of the sound frequency (or frequencies). The computing device, via the control device, may cause the plurality of output devices to output the sound frequency (or frequencies) associated with the selected one or more symptoms.

The plurality of output devices may be positioned proximate to the seating apparatus on which the user may be positioned. The arrangement of the plurality of output devices relative to the seating apparatus may provide for output of the sound frequency (or frequencies) associated with the selected one or more symptoms through one or more of the plurality of output devices that may be positioned near the user's head. The sound frequency (or frequencies) associated with the selected one or more symptoms may also be output through at least one output device of the plurality of output devices that may be positioned near the user's lower extremities. The at least one output device may be affixed or fastened to the seating apparatus such that the output of the sound frequency (or frequencies) associated with the selected one or more symptoms by the at least one output device may cause vibrations to pass through the seating apparatus. The output of the sound frequency (or frequencies) associated with the selected one or more symptoms by the plurality of output devices may provide health benefits for the user, such as alleviating the selected one or more symptoms.

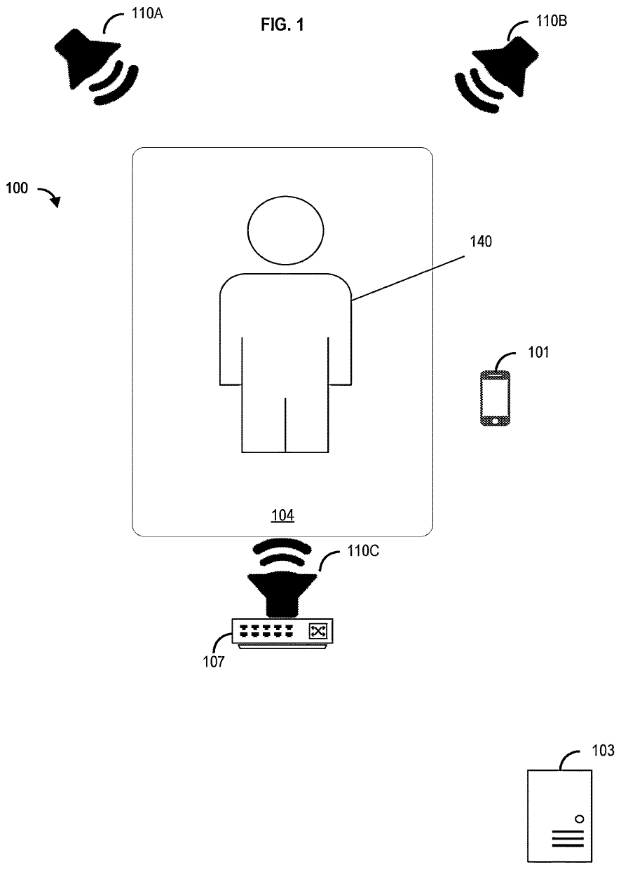

FIG. 1 shows an example system which may comprise a computing device 101, a seating apparatus 104, one of a plurality of output devices 110A-110C (e.g., first output device 110A, second output device 110B, and a third output device 110C), and a user 140. The computing device 101 may be a smartphone, a mobile device, a tablet, a laptop, or any other suitable computing device. The seating apparatus 104 may comprise, for example, an inflatable mattress, cushion, or similar implement capable of being filled with a gaseous medium (e.g., a low density, non-toxic gas, such as oxygen, helium, hydrogen, a combination thereof, and/or the like). In other examples, the seating apparatus 104 may be a non-inflatable seating apparatus, such as a mattress.

The system 100 may also comprise an application server 103. Though the application server 103 is shown with the user 140 and the seating apparatus 104 in FIG. 1, the application server 103 may be located in a remote location (e.g., in a different location that the user 140 and the seating apparatus 104). The application server 103 (also referred to herein as a “computing device(s)”) may be in communication with the computing device 101 and one or more of the plurality of output devices 110A-110C. As further described herein, the user 140 may be positioned upon the seating apparatus 104 and interact with the computing device 101 and/or the application server 103 to cause the surrounding output devices (e.g., 110A, 110B, and/or 110C) to output one or more of a plurality of sound frequencies.

The system 100 may also comprise a control deice 107. The control device 107 may comprise a stereo receiver, head unit, mixer, audio interface, a combination thereof, and/or the like. The control device 107 may be in communication with the plurality of output devices 110A-110C, the application server 103, and/or the computing device 101 via wired or wireless means. The control device 107 may provide power and/or signals to the plurality of output devices 110A-110C. In some examples, the functionality of the control device 107 may be controlled by the computing device 101 (e.g., via an application thereon) and/or by the application server 103.

Each output device of the plurality of output devices 110A-110C may comprise a stereo speaker, a monitor speaker, a subwoofer, a Bluetooth speaker, a smart speaker, one or components thereof, a combination thereof, and/or the like. The plurality of output devices (110A, 110B, and/or 110C) may each comprise one or more loudspeaker drivers, such as a “tweeter” that reproduces high frequency sounds, a “midrange” that reproduces mid-frequency sounds, and a “subwoofer” that reproduces low frequency sounds. The plurality of output devices (110A, 110B, and/or 110C) may each comprise a crossover network which divides input frequencies into two or more bands for their appropriate drivers. The plurality of output devices (110A, 110B, and/or 110C) may each comprise an acoustic cabinet that houses a loudspeaker driver(s). The examples for each of the plurality of output devices (110A, 110B, and/or 110C) described herein are meant to be exemplary only and not restrictive.

While the system 100 as shown in FIG. 1 includes three output devices 110A, 110B, 110C, it is to be understood that the system 100 may comprise more or less output devices and/or configurations of output devices with respect to the seating apparatus 104. While the system 100 as shown in FIG. 1 shows only one computing device 101, it is to be understood that the system 100 may comprise a plurality of computing devices configured to perform the methods described herein.

As described herein, the system 100 may be configured to output a plurality of sound frequencies proximate to the user 140 positioned on the seating apparatus 104 to provide health benefits to the user 140. In some examples, the first output device 110A and the second output device 110B may be positioned proximate to a superior portion of the seating apparatus 104 as shown in FIG. 1 such that output of sound (referred to herein also as sound frequency(ies) or output frequency(ies)) from the first output device 110A and/or the second output device 110B may be output relative to the where the user's 140 head is positioned. The system 100 may further provide for a separate output of sound by the third output device 110C that may differ (e.g., by frequency(ies), duration(s), etc.) from the output(s) by the first output device 110A and/or the second output device 110B. The orientation of the third output device 110C may be positioned proximate to a posterior position (e.g., a bottom, end, etc.) of the seating apparatus 104 such that output of sound by the third output device 110C may be relative to where the user's 140 feet or lower extremities are positioned. Other configurations are possible as well.

In order to utilize the system 100, the user 140 may interact with the system 100 via the computing device 101. As further described herein, the computing device 101 may receive a selection of a first symptom 160, output via a user interface, from the user 140. As further described herein, one or more sound frequencies and a duration(s) for each may be associated with the first symptom 160 in a database (e.g., application storage 142 described further herein). The computing device 101 may determine based on the first symptom 160, or cause the application server 103 to determine based on the first symptom 160, one or more sound frequencies and a duration(s) for each to be output via the first output device 110A, the second output device 110B, and/or 110C.

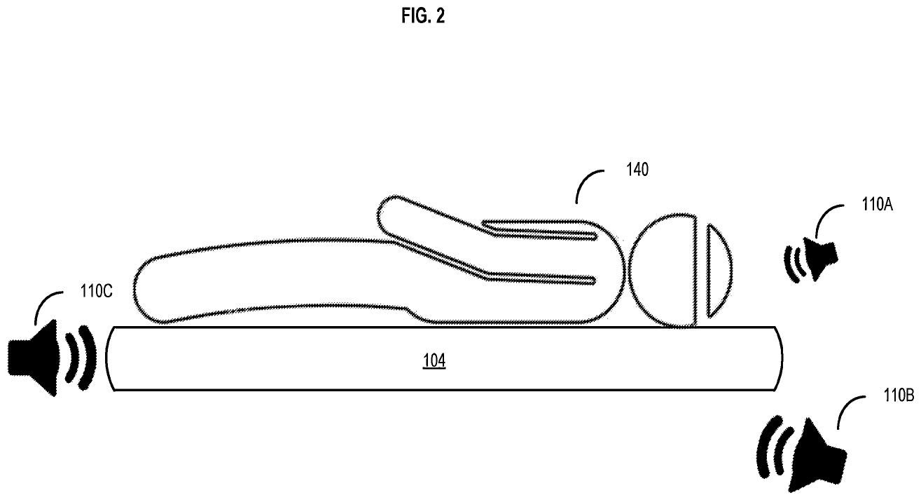

FIG. 2 shows a side view of system 100, including the seating apparatus 104, output device 110A, output device 110B, and output device 110C. In one example configuration of the system 100, the orientation of the seating apparatus 104 and output devices 110A, 110B, and 110C may be arranged as shown in FIG. 2, where the output devices 110A and 110B are positioned at the superior position of the seating apparatus 104 (e.g., near the user's 140 head) and the output device 110C is positioned at the posterior position of the seating apparatus 104 (e.g., near the user's 140 feet). Other configurations are possible as well. For example, one or both of the 110A and 110B may be positioned at or near a side of the seating apparatus 104 (e.g., near the user's 140 arms, waist, etc.). The output devices 110A and 110B may comprise, for example, monitor speakers. The output device 110C may comprise, for example, a subwoofer speaker. As described herein, the seating apparatus 104 may comprise an inflatable mattress capable of being filled with a gaseous medium (e.g., a low density, non-toxic gas, such as oxygen, helium, hydrogen, a combination thereof, and/or the like). The one or more sound frequencies output by the output devices 110A, 110B, 110C may excite (e.g., act upon) the gaseous medium within the seating apparatus 104. As discussed herein, the output device 110C may comprise a subwoofer speaker. In some example configurations, the output device 110C may be fastened/affixed to, or enclosed within, the seating apparatus 104. For example, the output device 110C may be fastened/affixed to, or enclosed within, the seating apparatus 104 in such a manner to excite the gaseous medium within the seating apparatus 104 and cause the user 140 to experience a series of vibrations and/or movements of the seating apparatus 104. The series of vibrations and/or movements of the seating apparatus 104 may differ depending upon the particular one or more sound frequencies (and duration(s) for each) that are output by the output device 110C (and/or any of the output devices 110A, 110B). The series of vibrations and/or movements of the seating apparatus 104 may provide therapeutic benefits for the user 140 as described herein to alleviate both physical and mental illness symptoms (e.g., corresponding to a symptom(s) indicated by the user 140 via the computing device 101).

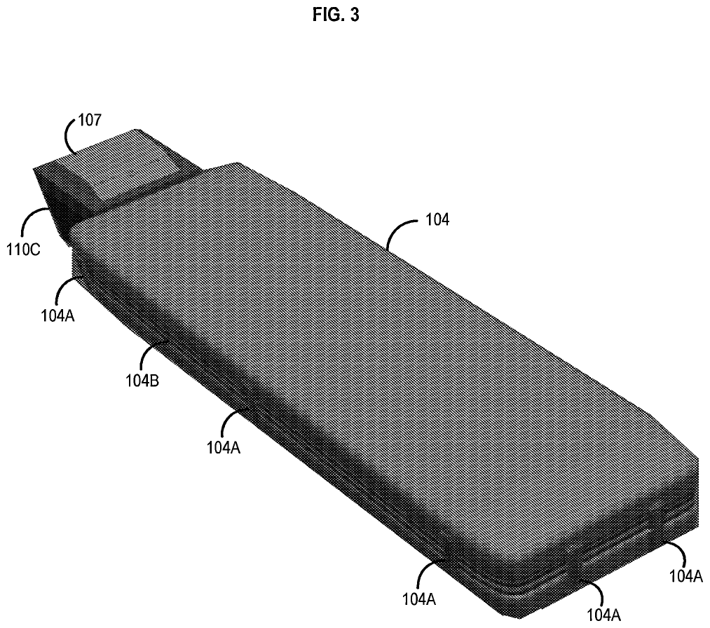

FIG. 3 shows a side view of system 100, including the seating apparatus 104, the output device 110C, and the control device 107. As described herein, the output device 110C may comprise a subwoofer speaker, and the output device 110C may be fastened/affixed to the seating apparatus 104. For example, as shown in FIG. 3, the output device 110C may comprise a housing that may be fastened/affixed to the seating apparatus 104 via one or more fastening elements 104A and a fastening element 104B. The fastening element 104B may comprise a cord(s), a strap(s), and/or any other suitable fastener(s) for fastening/affixing the housing of the output device 110C to the seating apparatus 104. The one or more fastening elements 104A may comprise buckles, straps, and/or any other suitable fastener(s) configured to assist with fastening/affixing the fastening element 104B to an outer perimeter of the seating apparatus 104. Other examples for fastening/affixing the housing of the output device 110C to the seating apparatus 104 are possible as well. The output device 110C may be fastened/affixed to the seating apparatus 104 such that the housing of the output device 110C may be in direct contact (or substantially in direct contact) with the seating apparatus 104 to enable the output device 110C to excite (e.g., via vibration) the gaseous medium within the seating apparatus 104 (and/or the seating apparatus 104 itself) when sound is output by the output device 110C.

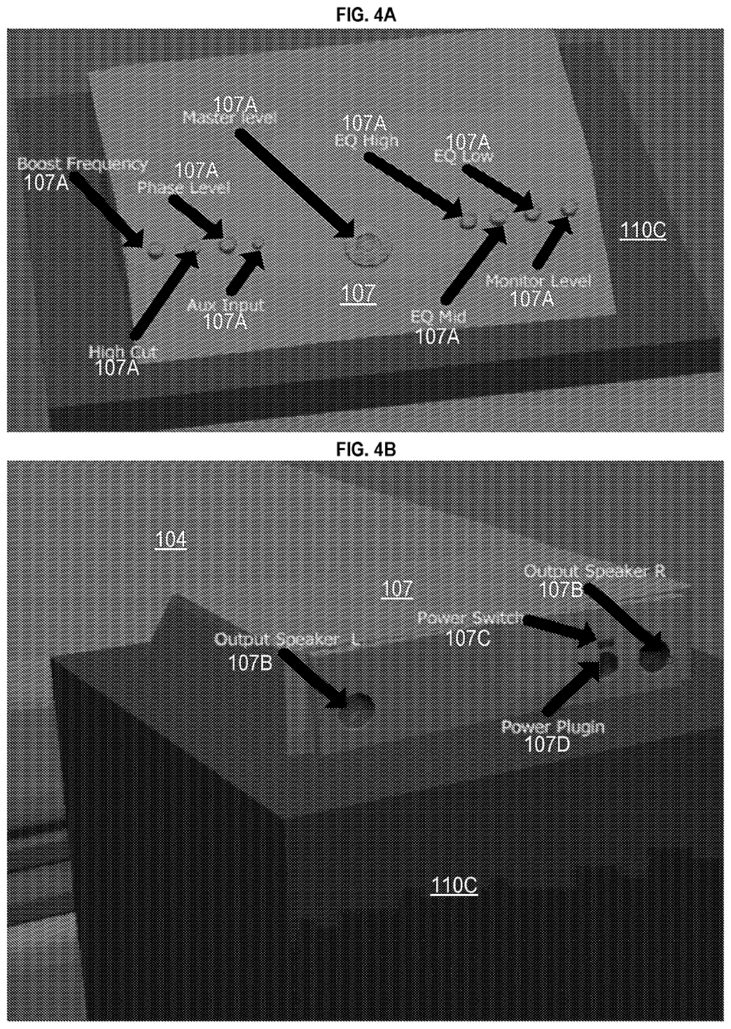

FIG. 4A shows an example top view of the control device 107 and the output device 110C. The control device 107 may comprise a stereo receiver, head unit, mixer, audio interface, a combination thereof, and/or the like. The control device 107 may be in communication with the plurality of output devices 110A-110C, the application server 103, and/or the computing device 101 via wired or wireless means. The control device 107 may provide power and/or signals to the plurality of output devices 110A-110C. As shown in FIG. 4A, the control device 107 may comprise a plurality of control elements 107A. The plurality of control elements 107A may each comprise a knob, switch, etc., for controlling output of sound by the plurality of output devices 110A-110C. For example, the plurality of control elements 107A may allow for adjustment or control of: boost frequency, phase level, high cut, an auxiliary input source, a master level, equalizer high, equalizer low, equalizer mid, a monitor level, etc. The examples for the plurality of control elements 107A described herein and shown in FIG. 4A are meant to be exemplary only and not restrictive. FIG. 4B shows an example rear view of the control device 107, the output device 110C, and the seating apparatus 104. As shown in FIG. 4B, the control device 107 may comprise a plurality of input elements 107B. The plurality of input elements 107B may each comprise an input element (e.g., an input jack) for one or more of the plurality of output devices 110A-110C. The control device 107 may further comprise a power switch 107C and a power plugin 107D (e.g., for a power cord). The examples described herein and shown in FIG. 4B are meant to be exemplary only and not restrictive.

In some examples, the methods described herein may be implemented using software, routines, rules, etc., existing entirely/natively on the computing device 101 (e.g., via one or more applications executing thereon). In other examples, the computing device 101 may be in communication with the application server 103 (e.g., via the one or more applications executing thereon), which may comprise some or all of the necessary software, routines, rules, etc., required to implement the methods described herein. The application server 103 may be part of, or associated with, a sound therapy system, program, etc. As further described herein, the user 140 may interact with a user interface 101A provided by the computing device 101 in order to communicate with the application server 103 and/or the computing device 101.

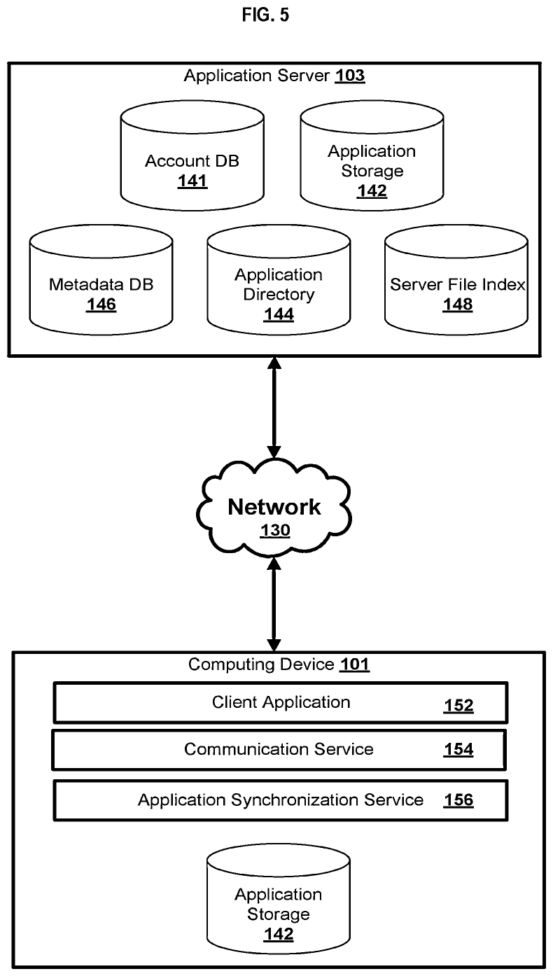

Turning now to FIG. 5, an expanded block diagram of the application server 103 and the computing device 101 are shown. The computing device 101 and the control device 107 may each be in communication with the application server 103 via a network 130. The application server 103 may have a plurality of storage mediums/databases, such as an account database 141, an application storage database 142, an application directory 144, a server file index 148, and/or a metadata database 146. The application server 103 may store application items in association with user accounts. The application server 103 may enable a user to access application item(s) from multiple user devices, such as the computing device 101, via the network 130 (e.g., Internet; cellular data networks, including 3G, LTE, etc.; wide area networks; local area networks; virtual networks, wireless networks, etc.). The network 130 may provide communication between the application server 103, the computing device 101, and the control device 107. For example, the computing device 101 may have a communication service 154 having a plurality of communication modules (e.g., a wireless receiver and/or transceiver, such as a WiFi module, a Bluetooth module, an antenna module). The computing device 101 may use the communication service 154 to communicate with the application server 103 via the network 130.

The application server 103 may support a plurality of accounts. A user(s) (e.g., the user 140) may create an account with the application server 103, and account details may be stored in an account database 141. The account database 141 may store profile information for registered users. In some cases, profile information for a registered user may include a username and/or email address. The account database 141 may include account management information, such as account type (e.g., administrator vs. normal user), security settings, personal configuration settings, etc.

The account database 141 may store groups of accounts associated with a user group. A user group may have permissions based on group policies and/or access control lists. For example, one user group (e.g., patients, medical professionals, etc.) may have access to one set of application items while another user group (e.g., patients, medical professionals, etc.) may have access to another set of application items. An administrator of a user group may modify groups, modify user accounts, etc. The application items may be stored in an application storage database 142. The application items may be any digital data such as documents, collaboration application items, text files, audio files, image files, video files, webpages, executable files, binary files, SQL queries, update messages, etc. For example, the application items stored in the application storage database 142 may comprise a plurality of symptoms, a plurality of sound frequencies, a plurality of rules (e.g., for output of the plurality of sound frequencies), etc. The plurality of rules may associate/relate each of the plurality of symptoms to one or more of the plurality of sound frequencies, etc. For example, one or more sound frequencies may be associated in the application storage database 142 with each of the plurality of symptoms. The one or more sound frequencies associated in the application storage database 142 with a given symptom of the plurality of symptoms may also be associated with a duration(s) defined by one of the plurality of rules. The plurality of rules may indicate or control the output of the one or more sound frequencies (e.g., which output device(s) to use, how long to output each sound at each output device, etc.). The plurality of rules may indicate or control a pattern(s) for the output of the one or more sound frequencies, such as in a pulse format, where a sound frequency is not played continuously (e.g., output of a sound frequency for one second, no output of a frequency for the next second, output of a sound frequency for one second, etc.).

The application storage database 142 may be combined with other types of storage mediums or databases to handle specific functions. The application storage database 142 may store application items, while metadata associated with the application items may be stored in a metadata database 146. The metadata associated with the application items may include one or more of a date, a time, a user identifier, a user device identifier, a changelog, and the like. Data identifying where an application item is stored in the application storage database 142 may be stored in an application directory 144. Additionally, data associated with changes, access, etc. may be stored in a server file index 148. Each of the various storage mediums/databases, such as the application storage database 142, the application directory 144, the server file index 148, and the metadata database 146 may include more than one such storage medium or database and may be distributed over many devices and locations. Other configurations are also possible. For example, data from the application storage database 142, the application directory 144, the server file index 148, and/or the metadata database 146 may be combined into one or more content storage mediums or databases or further segmented into additional storage mediums or databases. Thus, the application server 103 may include more or less storage mediums and/or databases than shown in FIG. 5.

The application storage database 142 may have software or other processor executable instructions for managing the storage of application items including, but not limited to, receiving application items for storage, preparing application items for storage, updating application items, selecting a storage location for an application item, retrieving application items from storage, etc. The application directory 144 may include an entry for each application item stored in the application storage database 142. The entry may be associated with a unique ID, which identifies an application item.

The application storage database 142 may also store metadata describing application items, application item types, and/or the relationship of application items to various user accounts, collections, or user groups in the metadata database 146, in association with the unique ID of the application item. The application storage database 142 may also store a log of data regarding changes, access, etc. (e.g., a changelog) in the server file index 148. The server file index 148 may include the unique ID of the application item and a description of the change or access action along with a time stamp or version number and any other relevant data.

The computing device 101 may have a client application 152 (e.g., the user interface 101A) stored thereon (e.g., in memory of the computing device 101). The client application 152 may provide front-end logic at the computing device 101 to enable a user of the computing device 101 to interact with the client application 152 to select at least one symptom of the plurality of symptoms, which may cause the output devices 110A, 110B, 110C to output at least one sound frequency of the plurality of sound frequencies (e.g., based on the plurality of rules that relate each of the plurality of symptoms to one or more of the plurality of sound frequencies).

The client application 152 may include an application item synchronization service 156. The application item synchronization service 156 may be in communication with the application storage database 142 to synchronize changes to application items between the computing device 101 and the application server 103. For example, as shown in FIG. 5, the application item synchronization service 156 may cause a local copy of the application storage database 142 to be stored at the computing device 101. In this way, the computing device 101 may access the application storage database 142 without being in communication with the application server 103. The computing device 101 may synchronize application items with the application server 103 via the application item synchronization service 156. Synchronization may be platform agnostic. That is, application items may be synchronized across multiple user devices of varying types, capabilities, operating systems, etc. The application synchronization service 156 may synchronize any changes (e.g., new, deleted, modified, copied, or moved application items) to application items in a designated location of a file system of the computing device 101.

The human body (e.g., the user's 140 body) has seven main energy centers, each center having its own frequency, energy, “brain,” hormones, and chemicals, and is controlled by the body's autonomic nervous system. The energy centers are:

1. Superior Mesenteric Plexus “Root”: this includes the spine, bladder, blood, kidneys, feet, and male reproductive organs of the human body;
2. Superior Plexus “Sacral”: this includes the lymphatic and circulatory systems, kidneys, adrenal glands, skin, and female reproductive organs of the human body;
3. Solar/Celiac Plexus: this includes the nervous system, stomach, gall bladder, large intestine, liver, and pancreas of the human body;
4. Heart Plexus: this includes the circulatory and respiratory systems, arms, hands, shoulders, ribs, breast, diaphragm, and thymus gland of the human body;
5. Thyroid Plexus “Throat”: this includes the thyroid, larynx, trachea, ears, nose, teeth, mouth, and throat of the human body;
6. Pineal Plexus “Third Eye”: this includes the pituitary gland, eyes, nose, ears, and skeletal system of the human body; and
7. Pituitary Plexus “Crown”: this includes the brain, nervous system, and pineal gland of the human body

Research suggests that electromagnetic field patterns determine physical and mental conditions of humans. For instance, human DNA is polar, meaning it has an uneven distribution of electron density. Because of this polarity, DNA can be manipulated with electrical and magnetic charges. Electrical charges are known as “thought/intention” while magnetic charges are known to be associated with “feelings.” Both electrical and magnetic charges combined would create a “coherent field.” It is believed that a coherent field creates an internal association between gratitude and the thought of health, where the creation of a coherent field synchronizes an individual's thought with how they feel. For instance, a coherent field will produce a positive effect on individuals, changing feelings of resentment to joy, frustration to freedom, and impatience to gratitude. Additionally, there is also an “incoherent field,” where an individual's thoughts are not aligned with that individual's “feelings,” thus creating a disassociation between health and thought.

When the autonomic nervous system is imbalanced, the human brain also becomes imbalanced, which impacts the human body's sympathetic nervous system. The body's sympathetic nervous system activates the body's fight or flight reaction, adding to increased stress on the body, resulting in the individual feeling physical and/or mental illness symptoms. Meanwhile, the body's parasympathetic nervous system conserves energy and regulates bodily functions. The present systems and methods serve to deactivate the user's 140 sympathetic nervous system when the user 140 is feeling mental and/or physical illness symptoms and activate the parasympathetic nervous system to recalibrate the user's 140 body and promote health.

As discussed above, there is a coherent field (association between thought and health) and an incoherent field (disassociation between thought and health). The incoherent field is associated with the body's (e.g., the user's 140) sympathetic nervous system, where an individual fails to reconcile their thoughts with how they are feeling, creating added stress on the body (e.g., the user's 140). This stress creates chemical imbalances and triggers unhealthy hormone production, driving the brain into a faster frequency, known as high range beta waves. The frequency range for these high range beta waves is between 12 and 30 Hz. In this state, the mind attempts to control and predict feelings and actions, thus overloading the brain, producing an incoherent field state, and preventing proper function. Such symptoms include dilated pupils, decreased saliva production, increased heart rate, the constriction of blood vessels and the increase in blood pressure, dilated bronchi causing the lungs to overwork, reduced stomach and intestine motility, reduced digestive enzyme secretion, released glucose to increase blood sugar, stimulated adrenal glands to secrete stress hormones, and a relaxed bladder, to name a few.

The discussed coherent field is associated with the body's (e.g., the user's 140) parasympathetic nervous system, where an individual (e.g., the user 140) is in a relaxed state and the previously mentioned energy centers become coherent or aligned with one another. Upon the balancing of the brain and the body (e.g., the user's 140), the brain is driven into high range alpha waves. These high range alpha waves are between 8 and 12 Hz. In this state, an individual experiences symptoms such as: body growth and repair, constricted pupils, increased saliva production, decreased heart rate, indirectly dilated blood vessels, constricted bronchi, decreased blood flow to skeletal muscles, increased stomach and intestine motility, increased blood flow to GI tract, and increased digestive enzyme secretion.

One purpose of the present systems and methods is to help transition the user 140 from an incoherent field (high range beta waves) to a coherent field (high range alpha waves) to allow for reduced stress, symptoms related thereto, and transition from an alert state to a relaxed state.

By outputting one or more sound frequencies (e.g., via the output devices 110A-110C and according to the plurality of rules described above) associated with a symptom(s) selected by the user 140 via the user interface 101A, the user's 140 energy centers may be activated, manipulated, changed, etc. For example, the one or more sound frequencies output by the output devices 110A, 110B, 110C may excite (e.g., act upon) the gaseous medium within the seating apparatus 104 and cause the user 140 to experience a series of vibrations and/or movements of the seating apparatus 104. The series of vibrations and/or movements of the seating apparatus 104 may activate, act upon, manipulate, change, etc. the user's 140 energy centers. For example, by providing the one or more sound frequencies to the user's 140 energy centers, a coherent message (e.g., the output frequency) may be sent (e.g., via the vibrations and/or movements of the seating apparatus 104) to each energy center, creating cohesion amongst the user's 140 various energy centers. By doing so, the user's 140 brain rhythm may be provided a stimulated, fixed frequency (also known as intermittent photic stimulation (IPS)). By locking the user's 140 brain into a provided stimulated frequency, photic driving may be created, which may provide for physiologic responses based on a locking of a brain rhythm and IPS. Thus, the physiologic and mental symptoms felt by the user 140 may be alleviated, and the user's 140 parasympathetic nervous system may be activated so to put the user 140 in a relaxed state, allowing for growth and repair of the user's 140 body.

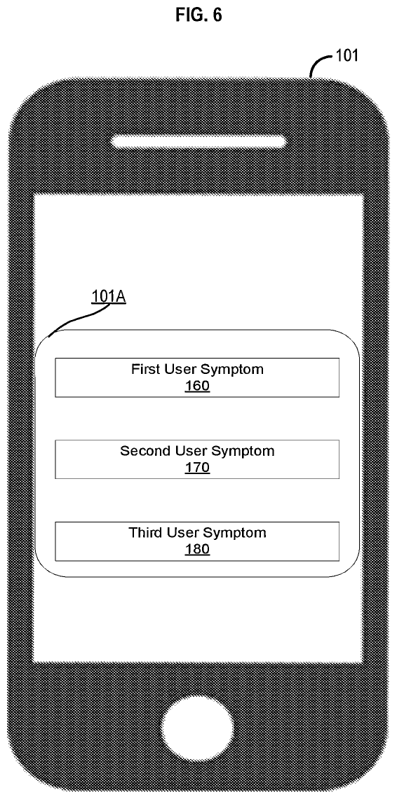

FIG. 6 shows an example user interface 101A of the computing device 101 displaying a plurality of user symptoms. The depicted user interface 101A may be a component/feature of the client application 152 described herein. The user interface 101A may display a plurality of user symptoms (e.g., a first user symptom 160, a second user symptom 170, a third user symptom 180, etc.) for the user 140 to select. The user interface 101A as depicted in FIG. 6 should not be limited in either orientation of the plurality of user symptoms or number of user symptoms displayed, but instead is merely an example of the configuration of the user interface 101A. For example, the first computing device 101 may output, at the user interface 101A, a listing of the plurality of user symptoms, ailments, etc. The user 140 may select, via the user interface 101A, one or more items (e.g., mental and/or physical symptoms he or she is experiencing, ailments, etc.) from the list, such as the first user symptom 160, the second user symptom 170, the third user symptom 180, etc. The computing device 101A may send an indication of the selection to the application server 103 second computing device(s) (e.g., a server, etc.). As described herein, the application server 103 may comprise an application storage database 142 storing the plurality of rules. The plurality of rules may be used by the application server 103 to determine which sound frequency, or plurality of sound frequencies, to cause the output device 110A, the output device 110B, and/or the output device 110C to output in response to the user's 140 selection from the list (e.g., the first user symptom 160, the second user symptom 170, the third user symptom 180, etc.). For example, the plurality of rules may be used by the application server 103 to determine which sound frequency, or plurality of sound frequencies, —as well as a pattern(s) and/or duration(s)—to cause the output device 110A, the output device 110B, and/or the output device 110C to output in response to the user's selection.

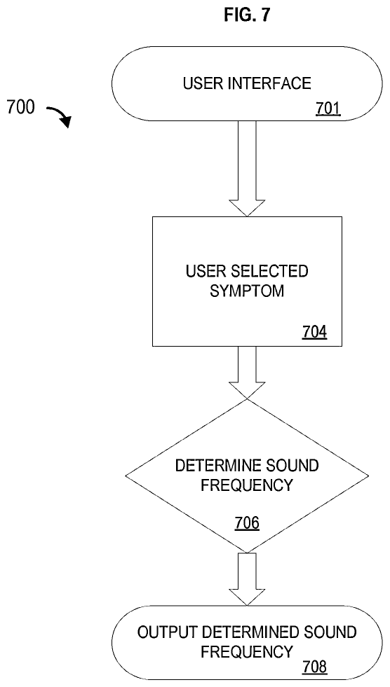

FIG. 7 shows an example flow diagram 700 for providing therapeutic sound treatment via the system 100. For example, the computing device 101 may be operated by the user 140 via the user interface 101A. At 701, the user 140 may be presented via the user interface 101A with a plurality of symptoms (e.g., the first user symptom 160, the second user symptom 170, the third user symptom 180, etc.). Once presented with a plurality of symptoms on the user interface 101A, at 704 the user 140 may select a symptom from among the plurality of symptoms presented via the user interface 101A. The selected symptom may comprise a physical and/or mental symptom that the user 140 may be experiencing. The computing device 101 may receive an indication of the selected symptom from 704.

At 706, the computing device 101 (or the application server 103) may determine, based on the selected symptom, at least one sound frequency of the plurality of sound frequencies to cause the output device 110A, the output device 110B, and/or the output device 110C to output. The computing device 101 may store the plurality of sound frequencies in relation to the stored plurality of symptoms via the application database 142. Based on the particular symptom(s) the user 140 selects, the computing device 101 may determine, based on the rules stored in the application database 142, which sound frequency, or plurality of sound frequencies to output as well as a pattern(s) and/or duration(s) for output.

The rules stored by the application database 142 may also include additional associations between the plurality of sound frequencies and plurality of symptoms. For instance, the computing device 101 may store rules via the application database 142 which provide for durations of time at which the sound frequencies are output, based on the user's 140 selected symptom(s). Additionally, the application database 142 may also store rules providing for a pattern(s) of the output of the associated sound frequency, such as in a pulse format, where the sound frequency is not played continuously (e.g., output of a sound frequency for one second, no output of a frequency for the next second, output of a sound frequency for one second, etc.) in relation to the user's 140 selection of their symptom. The computing device 101 may further provide for storing a user's 140 customized sound frequency in the application database 142. For instance, the user 140 may find that a specific sound frequency associated with a selected symptom provides greater health benefits when adjusted to the user's 140 preferences (i.e., pulses, output volume, duration of playing, etc.,) and wishes to use that customized sound frequency selection for alleviation of their symptoms in the future. By providing for the storage of such a customized sound frequency via the computing device 101 and application database 142, the user 140 may access said customized sound frequency at a later time. At 708, the computing device 101 (or the application server 103) may cause an output(s) of the at least one sound frequency. For example, the computing device 101 (or the application server 103) may via cause an output(s) of the at least one sound frequency via at least one of the output device 110A, 110B, or 110C.

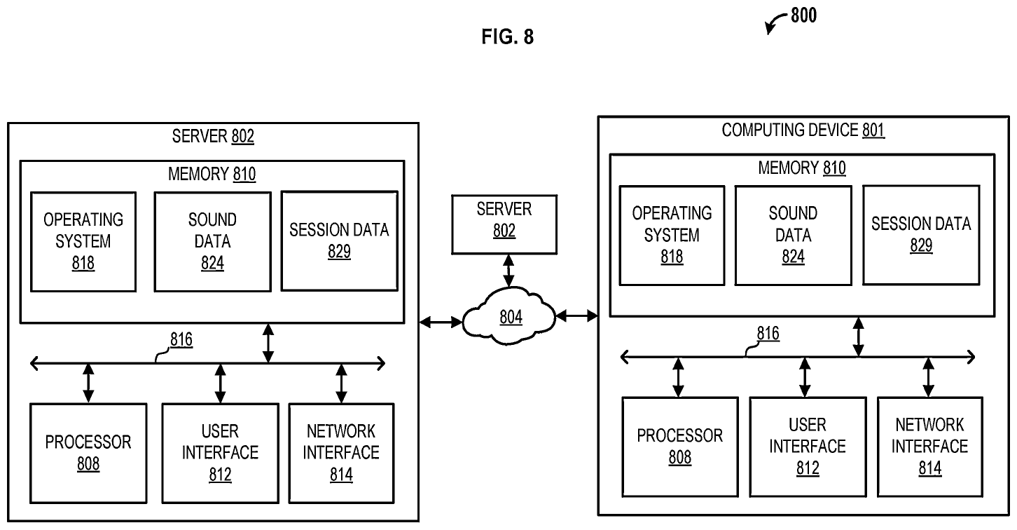

The present methods and systems may be computer-implemented. FIG. 8 shows a block diagram depicting a system/environment 800 comprising non-limiting examples of a computing device 801 and a server 802 connected through a network 804. Either of the computing device 801 or the server 802 may be any of the devices or components of the system 100 described herein. In an aspect, some or all steps of any described method may be performed on a computing device 801 as described herein. The computing device 801 may comprise one or multiple computers configured to store one or more of session data 829 and/or the like related to use of the computing device 801 and/or the server 802. The server 802 may comprise one or multiple computers configured to store sound data 824 (e.g., pitch, frequency, tone of a sound and related metadata). Multiple servers 802 may communicate with the computing device 801 via the network 804.

The computing device 801 and the server 802 may be a digital computer that, in terms of hardware architecture, generally includes a processor 808, system memory 810, user interfaces 812, and network interfaces 814. These components (808, 810, 812, and 814) are communicatively coupled via a local interface 816. The local interface 816 may be, for example, but not limited to, one or more buses or other wired or wireless connections, as is known in the art. The local interface 816 may have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 808 may be a hardware device for executing software, particularly that stored in system memory 810. The processor 808 may be any custom made or commercially available processor, a central processing unit (CPU), an auxiliary processor among several processors associated with the computing device 801 and the server 802, a semiconductor-based microprocessor (in the form of a microchip or chip set), or generally any device for executing software instructions. During operation of the computing device 801 and/or the server 802, the processor 808 may execute software stored within the system memory 810, to communicate data to and from the system memory 810, and to generally control operations of the computing device 801 and the server 802 pursuant to the software.

The user interface 812 may be used to receive user input from, and/or for sending system output to, one or more devices or components. For example, the user interface 812 may comprise the user interface 101A of the computing device 101. System output may be output via a display device and a printer (not shown). User interface 812 may include, for example, a serial port, a parallel port, a Small Computer System Interface (SCSI), an infrared (IR) interface, a radio frequency (RF) interface, and/or a universal serial bus (USB) interface.

The network interface 814 may be used to transmit and receive from the computing device 801 and/or the server 802 on the network 804. The network interface 814 may include, for example, a 10BaseT Ethernet Adaptor, a 10BaseT Ethernet Adaptor, a LAN PHY Ethernet Adaptor, a Token Ring Adaptor, a wireless network adapter (e.g., WiFi, cellular, satellite), or any other suitable network interface device. The network interface 814 may include address, control, and/or data connections to enable appropriate communications on the network 804.

The system memory 810 may include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the system memory 810 may incorporate electronic, magnetic, optical, and/or other types of storage media. Note that the system memory 810 may have a distributed architecture, where various components are situated remote from one another, but may be accessed by the processor 808.

The software in system memory 810 may include one or more software programs, each of which comprises an ordered listing of executable instructions for implementing logical functions. In the example of FIG. 8, the software in the system memory 810 of the computing device 801 may comprise the session data 829, the sound data 824, and a suitable operating system (O/S) 818. In the example of FIG. 8, the software in the system memory 810 of the server 802 may comprise the session data 829, the sound data 824, and a suitable operating system (O/S) 818. The operating system 818 essentially controls the execution of other computer programs and enables scheduling, input-output control, file and data management, memory management, and communication control and related services.

For purposes of illustration, application programs and other executable program components such as the operating system 818 are shown herein as discrete blocks, although it is recognized that such programs and components may reside at various times in different storage components of the computing device 801 and/or the server 802. An implementation of the system/environment 800 may be stored on or transmitted across some form of computer readable media. Any of the disclosed methods may be performed by computer readable instructions embodied on computer readable media. Computer readable media may be any available media that may be accessed by a computer. By way of example and not meant to be limiting, computer readable media may comprise “computer storage media” and “communications media.” “Computer storage media” may comprise volatile and non-volatile, removable and non-removable media implemented in any methods or technology for storage of information such as computer readable instructions, data structures, program modules, or other data. Exemplary computer storage media may comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by a computer.

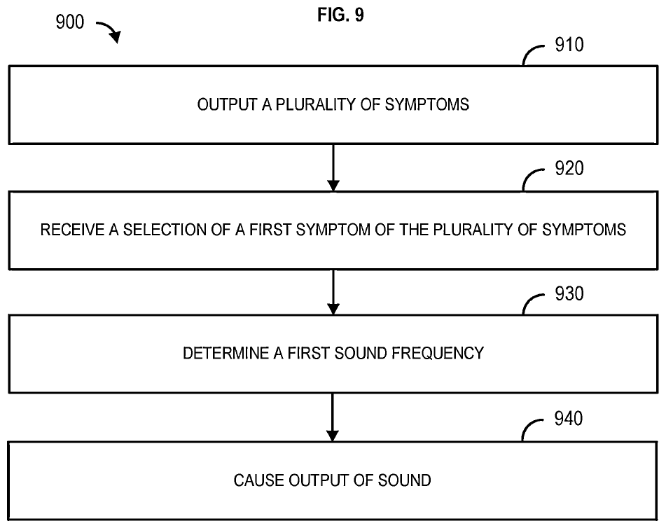

FIG. 9 shows a flowchart of an example method 900 for providing therapeutic sound treatment. The method 900 may be performed in whole or in part by a single computing device, a plurality of computing devices, and the like. For example, some or all steps of the method 900 may be performed by at least one of the computing devices shown in FIG. 8. For ease of explanation, the steps of the method 900 are described herein as being performed by a single computing device. However, it is to be understood that some steps of the method 900 may be performed by a first computing device, while other steps of the method 900 may be performed by another computing device(s). For example, the computing device may comprise a mobile device, such as the computing device 101.

At step 910, the computing device (e.g., the computing device 101, 801, etc.) may output a plurality of symptoms. For example, the computing device may output the plurality of symptoms via a user interface (e.g., a display, screen, etc.) of the computing device (e.g., user interface 101A). The plurality of symptoms may be associated with a plurality of sound frequencies. For example each symptom of the plurality of symptoms may be associated with one or more frequencies of the plurality of sound frequencies. At step 920, the computing device may receive a selection of a first user symptom of the plurality of symptoms (e.g., the first user symptom 160). For example, the computing device may receive the selection of the first user symptom from a user (e.g., the user 140) of the computing device. The user may be situated on, or proximate to, a seating apparatus (e.g., the seating apparatus 104). The user may make the selection via the user interface.

At step 930, the computing device may determine a first sound frequency. For example, the computing device may comprise sound data (e.g., the sound data 824) stored in memory, and the sound data may indicate that the first sound frequency corresponds to the first user symptom. At step 940, the computing device may cause output of sound. The sound may be output by the computing device at the first sound frequency. The computing device may output the sound via at least one output device (e.g., 110A, 110B, and/or 110C). For example, the computing device may be in communication with, via a control device (e.g., the control device 107), the at least one output device (e.g., 110A, 110B, and/or 110C). The computing device may cause, via the control device, the at least one output device to output the first sound.

The at least one output device may output the first sound frequency for a first duration of time. For example, the computing device may determine the first duration of time. The computing device may determine the first duration of time based on the first user symptom, the first sound frequency, a combination thereof, and/or the like. The computing device may determine the first duration of time in response to receiving of the selection of the first symptom at step 920. The computing device may cause output of a second sound frequency. The computing device may cause output of the second sound frequency in response to receiving the selection of the first user symptom at step 920. The second sound frequency may be output through at least one of the plurality of output devices (e.g., output device 110A, 110B, or 110C). For example, the second sound frequency may be output via the at least one output device, another output device, a combination thereof, and/or the like. The second sound frequency may comprise a higher pitched frequency as compared to the first sound frequency or vice-versa. The higher pitched sound frequency of the two may be output through an output device (e.g., the output device 110A and/or 110B) positioned at a superior portion of the seating apparatus, and the lower pitched frequency of the two may be output through an output device positioned at a posterior end of seating apparatus.

Additionally, or in the alternative, the computing device (e.g., 101) may provide the user with a selectable option(s) (e.g., via the user interface) to select more than one of the plurality of symptoms for the outputting of more than one sound frequency via more than one of the plurality of output devices 110A, 110B, and/or 110C. Each sound frequency selected for output may be output simultaneously, consecutively, randomly, etc. For example, the computing device may cause each sound frequency selected to be output simultaneously, consecutively, randomly, etc. Other examples are possible as well.

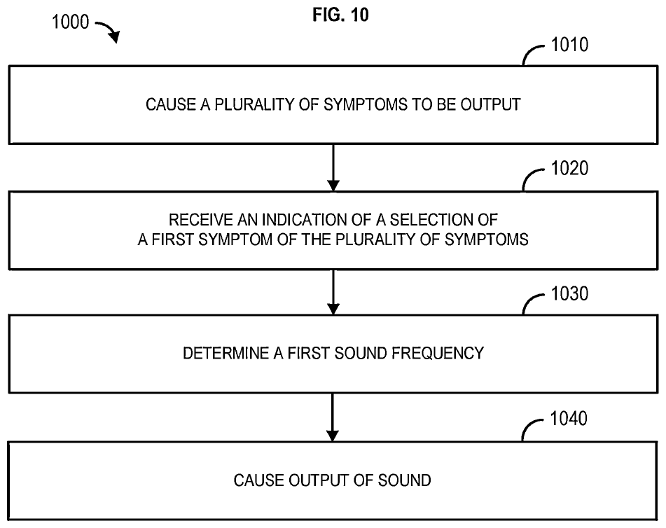

FIG. 10 shows a flowchart of an example method 1000 for providing therapeutic sound treatment. The method 1000 may be performed in whole or in part by a single computing device, a plurality of computing devices, and the like. For example, some or all steps of the method 1000 may be performed by at least one of the computing devices 801 shown in FIG. 8. For ease of explanation, the steps of the method 1000 are described herein as being performed by a single computing device. However, it is to be understood that some steps of the method 1000 may be performed by a first computing device, while other steps of the method 1000 may be performed by another computing device(s). For example, the computing device may comprise an application server (e.g., the application server 103) in communication with a mobile device, such as the computing device 101.

At step 1010, the computing device may cause the mobile device (e.g., the computing device 101, 801, etc.) to output a plurality of symptoms. For example, the computing device may cause the mobile device to output the plurality of symptoms via a user interface (e.g., a display, screen, etc.) of the mobile device (e.g., user interface 101A). The plurality of symptoms may be associated with a plurality of sound frequencies. For example each symptom of the plurality of symptoms may be associated with one or more frequencies of the plurality of sound frequencies. At step 1020, the computing device may receive an indication of a selection of a first user symptom of the plurality of symptoms (e.g., the first user symptom 160). For example, the mobile device may receive a selection of the first user symptom from a user (e.g., the user 140) of the mobile device. The user may be situated on, or proximate to, a seating apparatus (e.g., the seating apparatus 104). The user may make the selection via the user interface of the mobile device. The mobile device may send the indication of the selection of the first user symptom to the computing device.

At step 1030, the computing device may determine a first sound frequency. For example, the computing device may comprise sound data (e.g., the sound data 824) stored in memory, and the sound data may indicate that the first sound frequency corresponds to the first user symptom. At step 1040, the computing device may cause output of sound. The sound may be output at the first sound frequency. For example, the computing device may be in communication with, via a control device (e.g., the control device 107), at least one output device (e.g., 110A, 110B, and/or 110C). The computing device, via the control device, may cause the at least one output device to output the first sound.

The at least one output device may output the first sound frequency for a first duration of time. For example, the computing device may determine the first duration of time. The computing device may determine the first duration of time based on the first user symptom, the first sound frequency, a combination thereof, and/or the like. The computing device may determine the first duration of time in response to receiving of the indication of the selection of the first symptom at step 1020. The computing device may cause a second sound frequency to be output. The computing device may output the second sound frequency in response to receiving the selection of the first user symptom at step 520. The second sound frequency may be output through at least one of the plurality of output devices (e.g., output device 110A, 110B, or 110C). For example, the second sound frequency may be output via the at least one output device, another output device, a combination thereof, and/or the like. The second sound frequency may comprise a higher pitched frequency as compared to the first sound frequency or vice-versa. The higher pitched sound frequency of the two may be output through an output device (e.g., the output device 110A and/or 110B) positioned at a superior portion of the seating apparatus, and the lower pitched frequency of the two may be output through an output device positioned at a posterior end of seating apparatus.

Additionally, or in the alternative, the computing device (e.g., 101 or 301) may cause the mobile device to provide the user with a selectable option(s) (e.g., via the user interface) to select more than one of the plurality of symptoms for the outputting of more than one sound frequency via more than one of the plurality of output devices 110A, 110B, and/or 110C. Each sound frequency selected for output may be output simultaneously, consecutively, randomly, etc. For example, the computing device may cause each sound frequency selected to be output simultaneously, consecutively, randomly, etc. Other examples are possible as well.

While specific configurations have been described, it is not intended that the scope be limited to the particular configurations set forth, as the configurations herein are intended in all respects to be possible configurations rather than restrictive. Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

1. A method comprising:
causing, by a first computing device, a second computing device to output a plurality of symptoms at a user interface of the second computing device, wherein each symptom of the plurality of symptoms is associated with one or more frequencies of a plurality of sound frequencies;
receiving, from the second computing device, an indication of a selection of a first symptom of the plurality of symptoms, wherein the selection is made via the user interface;
determining, based on the indication and the first symptom, a first sound frequency of the plurality of sound frequencies; and
causing at least one output device to output the first sound frequency.

2. The method of claim 1, wherein the first computing device comprises an application server.

3. The method of claim 1, wherein the second computing device comprises a mobile device.

4. The method of claim 1, further comprising: receiving, by the second computing device, via the user interface, the selection of the first symptom.

5. The method of claim 1, further comprising: determining, based on at least one of the first symptom or the first sound frequency, a duration of time for outputting the first sound frequency.

6. The method of claim 5, wherein causing the at least one output device to output the first sound frequency comprises: causing the at least one output device to output the first sound frequency for the duration of time.

7. The method of claim 1, wherein the at least one output device comprises at least one speaker proximate to a seating apparatus.

8. The method of claim 7, wherein the at least one speaker is fastened to the seating apparatus.

9. The method of claim 7, wherein the seating apparatus comprises an inflatable mattress.

10. The method of claim 7, wherein the seating apparatus comprises a non-inflatable mattress.

11. A non-transitory computer-readable storage medium comprising computer-executable instructions that, when executed by a first computing device, cause the first computing device to:
cause a second computing device to output a plurality of symptoms at a user interface, wherein each symptom of the plurality of symptoms is associated with one or more frequencies of a plurality of sound frequencies;
receive, from the second computing device, an indication of a selection of a first symptom of the plurality of symptoms, wherein the selection is made via the user interface;
determine, based on the indication and the first symptom, a first sound frequency of the plurality of sound frequencies; and
cause at least one output device to output the first sound frequency.

12. The non-transitory computer-readable storage medium of claim 11, wherein the first computing device comprises an application server.

13. The non-transitory computer-readable storage medium of claim 11, wherein computing device comprises a mobile device.

14. The non-transitory computer-readable storage medium of claim 11, wherein the computer-executable instructions further cause the first computing device to receive, by the second computing device, via the user interface, the selection of the first symptom.

15. The non-transitory computer-readable storage medium of claim 11, wherein the computer-executable instructions further cause the first computing device to determine, based on at least one of the first symptom or the first sound frequency, a duration of time for outputting the first sound frequency.

16. The non-transitory computer-readable storage medium of claim 15, wherein the computer-executable instructions that cause the at least one output device to output the first sound frequency further cause the at least one output device to output the first sound frequency for the duration of time.

17. The non-transitory computer-readable storage medium of claim 11, wherein the at least one output device comprises at least one speaker proximate to a seating apparatus.

18. The non-transitory computer-readable storage medium of claim 17, wherein the at least one speaker is fastened to the seating apparatus.

19. The non-transitory computer-readable storage medium of claim 17, wherein the seating apparatus comprises a non-inflatable mattress.

20. The non-transitory computer-readable storage medium of claim 17, wherein the seating apparatus comprises an inflatable mattress.
